(19) United States
(12) Patent Application Publication
(10) Pub. No.: US 2026/0256219 A1
(43) Pub. Date: Sep. 3, 2026

(54) HIGH ABSORBENCY GARMENT LAYER AND METHOD OF MANUFACTURING SAME

(71) Applicant: THINX INC., Neenah, WI (US)

(72) Inventors: Courtney Newman, Beacon, NY (US); Oxana Razdolski, Franklin Lakes, NJ (US); Joyce Baran, Stratford, CT (US); Alexandra McNutt, Brooklyn, NY (US); Matt Westergren-kelly, New York, NY (US); Christen Graham, New York, NY (US)

(73) Assignee: THINX INC., Neenah, WI (US)

(21) Appl. No.: 18/835,543

(22) PCT Filed: Feb. 1, 2023
(86) PCT No.: PCT/US2023/012090

§ 371 Date: Aug. 2, 2024

Related U.S. Application Data
(63) Continuation-in-part of application No. 17/592,460, filed on Feb. 3, 2022.
(60) Provisional application No. 63/406,223, filed on Sep. 13, 2022.

(51) Int. Cl. A41B 9/00; A41B 9/08; A41D 1/089; A41D 31/02; A41D 31/12

(52) U.S. Cl. CPC A41B 9/001; A41B 9/004; A41B 9/08; A41D 1/089; A41D 31/02; A41D 31/125; A41B 2400/60; A41B 2500/00

(57) ABSTRACT

A garment including a body, two leg openings, and a crotch area on an inner side of the garment. The crotch area comprises: a first layer, a second layer, a third layer and a fourth layer, the first layer is an innermost layer, the fourth layer is an outermost layer, and the second and third layers are intermediate layers extending between the first and fourth layers. At least one of the first to fourth layers comprises a moisture-wicking material or a waterproof material, and each of the first to fourth layers is, independently, a single layer or a plurality of layers.

### CROSS REFERENCE TO RELATED APPLICATIONS

This patent application claims the benefit of U.S. Provisional Patent Application 63/406,223 filed 13 Sep. 2022, which is incorporated by reference herein for all purposes

This patent application claims the benefit of U.S. Nonprovisional patent application Ser. No. 17/592,460 filed 3 Feb. 2022, which is incorporated by reference herein for all purposes.

### TECHNICAL FIELD

This disclosure relates to a garment including a crotch area, which includes a moisture and/or liquid absorbable garment layer having high absorbency, and a method of manufacturing same.

### BACKGROUND

In this disclosure, where a document, an act, and/or an item of knowledge is referred to and/or discussed, then such reference and/or discussion is not an admission that the document, the act, and/or the item of knowledge and/or any combination thereof was at a priority date, publicly available, known to a public, part of common general knowledge, and/or otherwise constitutes any prior art under any applicable statutory provisions; and/or is known to comprise relevant to any attempt to solve any problem with which this disclosure is concerned with. Further, nothing is disclaimed.

There is a desire to enable a garment that is sufficiently waterproof and/or includes moisture-wicking properties, has high absorbency, and is adequately resistant to wear and tear. However, despite various attempts by others, this type of garment does not yet exist. As such, this disclosure enables such type of garment, including all possible combinations of any specific features described herein.

### SUMMARY

This disclosure at least partially addresses at least one of above inefficiencies. However, this disclosure can prove useful to other technical areas. Therefore, various claims recited below should not be construed as necessarily limited to addressing any of the above inefficiencies.

According to an exemplary embodiment of this disclosure, a device comprises: a garment comprising a body, two leg openings, and a crotch area on an inner side of the garment, wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: a garment comprising a body, two leg openings, a material surrounding each of the leg openings and having a length not extending past a knee of a wearer, and a crotch area on an inner side of the garment, wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: a garment comprising a body, two leg openings, a crotch area on an inner side of the garment, and a material surrounding each of the two leg openings and having a length extended past a knee of a wearer, wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: an outer garment surrounding an inner garment, wherein the outer garment extends beyond the inner garment, wherein the inner garment comprises a body, two leg openings, a crotch area on an inner side of the garment, an wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: a garment, wherein the garment comprises a front portion, wherein the front portion comprises a first front portion substantially covering a chest of a wearer, a second front portion substantially covering a torso of a wearer between the chest and a waistline of the wearer, and a third front portion substantially covering the torso of the wearer below the waistline; a rear portion; an inner front elastic portion connecting the first front portion and the second front portion at an inner side of the garment; an inner rear elastic portion across a back of the wearer to connect a first edge and a second edge of the first portion; two leg openings, and optionally, a pair of straps connecting the front portion and the rear portion; wherein the third portion comprises a crotch area, and wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

The device according to any one of the prior exemplary embodiments, wherein the second layer extends between the first layer and the third layer, and the third layer extends between the second layer and the fourth layer.

The device according to any one of the prior exemplary embodiments, wherein a material of each of the first to fourth layers is same or different, and is independently selected from fabric, natural yarn, synthetic yarn, cotton, silk, polyester, spandex, rubber, plastic, metal, merino wool, nylon, polypropylene, rayon, linen, spandex, bamboo, Gore-Tex, X-static, tencel or combinations thereof.

The device according to any one of the prior exemplary embodiments, further comprising one or more additional layers between the first and fourth layers.

The device according to any one of the prior exemplary embodiments, wherein two or more of the first to fourth layers are bonded or adhered together via a stitching, a knitting, an adhesive layer or a bonding tape.

The device according to any one of the prior exemplary embodiments, further comprising a fifth layer extending between the fourth layer and the main body, wherein the fifth layer is a single layer or a plurality of layers.

The device according to any one of the prior exemplary embodiments, wherein the second layer and the third layer are separate layers.

The device according to any one of the prior exemplary embodiments, wherein the second layer and third layer are bonded or adhered together via a stitching, a knitting, an adhesive layer or a bonding tape.

The device according to any one of the prior exemplary embodiments, wherein the second layer and the third layer are combined in a single layer.

The device according to any one of the prior exemplary embodiment,, wherein the second layer further comprises a pocket; and wherein the third layer is included inside the pocket.

The device according to any one of the prior exemplary embodiments, wherein the second layer further comprises a pocket; and wherein a plurality of third layers are included inside the pocket.

The device according to any one of the prior exemplary embodiments, wherein the third layer comprises one or more layers.

The device according to any one of the prior exemplary embodiments, wherein a material of each of the one or more layers is same or different, and is independently selected from fabric, natural yarn, synthetic yarn, cotton, silk, polyester, spandex, rubber, plastic, metal, merino wool, nylon, polypropylene, rayon, linen, spandex, bamboo, Gore-Tex, X-static, tencel or combinations thereof.

The device according to any one of the prior exemplary embodiments, wherein the moisture-wicking materials is selected from fabric, natural yarn, synthetic yarn, wool, polypropylene, nylon, polyester or combinations thereof.

The device according to any one of the prior exemplary embodiments, wherein a shape of the second layer is different from a shape of the third layer.

The device according to any one of the prior exemplary embodiments, wherein a shape of the second layer is the same as a shape of the third layer.

The device according to any one of the prior exemplary embodiments, wherein a size of the third layer is smaller than a size of the second layer such that the second layer extends beyond the third layer.

The device according to any one of the prior exemplary embodiments, wherein each of the first to fourth layers has a first shape, wherein an upper portion and a lower portion having equal dimensions are connected by a middle portion having a narrower width than the upper and lower portions.

The device according to any one of the prior exemplary embodiments, wherein each of the first to fourth layers has a modified shape, wherein the modified shape comprises a first shape portion connected to a second shape portion, wherein the first shape portion comprises an upper portion and a lower portion having equal dimensions connected by a middle portion having a narrower width than the upper and lower portions, and wherein the second shape portion comprises a trapezoidal shape tapering from the first shape portion to a rear edge of each of the first to fourth layers.

The device according to any one of the prior exemplary embodiments, wherein each of the first to fourth layers is arranged such that the first shape portion extends towards a front of the garment and the second shape portion extends towards a rear of the garment.

The device according to any one of the prior exemplary embodiments, wherein at least the first layer comprises the moisture-wicking material.

The device according to any one of the prior exemplary embodiments, wherein at least the first layer comprises the waterproof material.

The device according to any one of the prior exemplary embodiments, wherein the stitching, the knitting, the adhesive layer or the bonding tape avoids extending through the material.

The device according to any one of the prior exemplary embodiments, wherein each of the two leg openings is a free cut leg opening.

The device according to any one of the prior exemplary embodiments, wherein an elastic edge surrounds each of the two leg openings.

The device according to any one of the prior exemplary embodiments, wherein an elastic edge surrounds each of the two leg openings, and wherein an edge of each of the two leg openings is folded inwards to enclose the elastic edge.

The device according to any one of the prior exemplary embodiments, wherein an elastic edge portion surrounds a waistband portion of the body.

The device according to any one of the prior exemplary embodiments, wherein the first layer is bonded or adhered to the second layer along outer edges of the first and second layers via a stitching, a knitting, an adhesive layer or a bonding tape.

The device according to any one of the prior exemplary embodiments, wherein the crotch area is bonded to the body via a stitching, a knitting, an adhesive layer or a bonding tape, and wherein the stitching, the knitting, the adhesive layer or the bonding tape extends beyond the crotch area to a waistband edge of the body.

The device according to any one of the prior exemplary embodiments, wherein a grain of the moisture-wicking material is arranged to be longitudinal from a front to a rear portion of the at least one of the first to fourth layers comprising the moisture-wicking material.

The device according to any one of the prior exemplary embodiments, wherein a volume capacity of the one or more layers comprising the moisture-wicking material or waterproof material is in a range of from about 50 ml to about 300 ml.

The device according to any one of the prior exemplary embodiments, further comprising a material surrounding each of the leg openings and having a length extending past a knee of a wearer.

The device according to any one of the prior exemplary embodiments, further comprising a front waistband pocket located on an inner side of the garment.

The device according to any one of the prior exemplary embodiments, further comprising a temperature pad, such as, for example, a heating pad or a cooling pad inside the front waistband pocket.

The device according to any one of the prior exemplary embodiments, further comprising a mesh liner on an inner side of the body, and connected to the body along one edge.

The device according to any one of the prior exemplary embodiments, further comprising a pocket located in an inner side of a waistband portion, wherein the pocket includes a zipper, a self-fastener, an interconnecting fabric connector, a hook and loop fastener, a button fastener, a closure means, or a combination thereof.

The device according to any one of the prior exemplary embodiments, further comprising a front waistband pocket located on an inner side of the garment.

The device according to any one of the prior exemplary embodiments, further comprising a temperature pad inside the front waistband pocket.

The device according to any one of the prior exemplary embodiments, further comprising a mesh liner on an inner side of the body, and connected to the body along one edge.

The device according to any one of the prior exemplary embodiments, wherein an outer garment comprises a waist portion, a front first portion, a front second portion, a rear first portion, and a rear second portion, wherein the front first portion and second portion have a trim along edges thereof.

According to an exemplary embodiment of this disclosure, a method comprises: causing a garment to comprise a body, two leg openings, and a crotch area on an inner side of the garment, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, causing at least one of the first to fourth layers to comprise a moisture-wicking material or a waterproof material, and causing each of the first to fourth layers to be, independently, a single layer or a plurality of layers.

According to another exemplary embodiment of this disclosure, a method comprises: causing a garment to comprise a body, two leg openings, a material surrounding each of the leg openings and having a length not extending past a knee of a wearer, and a crotch area on an inner side of the garment, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, and causing at least one of the first to fourth layers comprises a moisture-wicking material or a waterproof material, and wherein each of the first to fourth layers is, independently, a single layer or a plurality of layers.

According to another exemplary embodiment of this disclosure, a method comprises: causing a garment to comprise a body, two leg openings, a crotch area on an inner side of the garment, and a material surrounding each of the two leg openings and having a length extended past a knee of a wearer, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, causing at least one of the first to fourth layers to comprise a moisture-wicking material or a waterproof material, and causing each of the first to fourth layers to be, independently, a single layer or a plurality of layers.

According to another exemplary embodiment of this disclosure, a method comprises: causing an outer garment to surround an inner garment, wherein the outer garment extends beyond the inner garment, causing the inner garment to comprise a body, two leg openings, a crotch area on an inner side of the garment, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, causing at least one of the first to fourth layers to comprise a moisture-wicking material or a waterproof material, and causing each of the first to fourth layers to be, independently, a single layer or a plurality of layers.

According to another exemplary embodiment of this disclosure, a method comprises: causing a garment to comprise a front portion, wherein the front portion comprises a first front portion substantially covering a chest of a wearer, a second front portion substantially covering a torso of a wearer between the chest and a waistline of the wearer, and a third front portion substantially covering the torso of the wearer below the waistline; causing a garment to comprise a rear portion; causing a garment to comprise an inner front elastic portion connecting the first front portion and the second front portion at an inner side of the garment; causing a garment to comprise an inner rear elastic portion across a back of the wearer to connect a first edge and a second edge of the first portion; causing a garment to comprise two leg openings; causing a garment to comprise, optionally, a pair of straps connecting the front portion and the rear portion; causing the third portion to comprise a crotch area; causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers; causing at least one of the first to fourth layers to comprise a moisture-wicking material or a waterproof material; and causing each of the first to fourth layers to be, independently, a single layer or a plurality of layers.

This disclosure is embodied in various forms illustrated in a set of accompanying illustrative drawings. Note that variations are contemplated as being a part of this disclosure, limited only by a scope of various claims recited below.

### DESCRIPTION OF DRAWINGS

The set of accompanying illustrative drawings shows various example embodiments of this disclosure. Such drawings are not to comprise construed as necessarily limiting this disclosure. Like numbers and/or similar numbering scheme can refer to like and/or similar elements throughout.

FIGS. 1A to 1D show various perspective views of a garment according to a first exemplary embodiment.

FIGS. 2A and 2B illustrate arrangements of various layers of a gusset according to exemplary embodiments.

FIG. 3 is an image of the arrangement of various layers of a gusset according to an exemplary embodiment.

FIGS. 4A to 4C show various perspective (outer) views of a garment according to a second exemplary embodiment.

FIGS. 5A to 5D show various perspective (inner) views of a garment according to a second exemplary embodiment.

FIGS. 6A to 6C are images of a garment according to a second exemplary embodiment.

FIGS. 7A to 7E show various perspective views of a garment according to a third exemplary embodiment.

FIGS. 8A to 8C are images of a garment according to a third exemplary embodiment.

FIGS. 9A to 9E show various perspective views of a garment according to a fourth exemplary embodiment.

FIGS. 10A to 10C are images of a garment according to a fourth exemplary embodiment.

FIGS. 11A to 11F show various perspective views of a garment according to a fifth exemplary embodiment.

FIGS. 12A to 12C are images of a garment according to a fifth exemplary embodiment.

FIG. 13 is an embodiment of garment including a gusset structure.

FIG. 14 is an enlarged view of the garment including a gusset structure of FIG. 13.

### DETAILED DESCRIPTION OF PREFERRED EMBODIMENTS

This disclosure is now described more fully with reference to the set of accompanying illustrative drawings, in which example embodiments of this disclosure are shown. This disclosure can be embodied in many different forms and should not be construed as necessarily being limited to the example embodiments disclosed herein. Rather, the example embodiments are provided so that this disclosure is thorough and complete, and fully conveys various concepts of this disclosure to those skilled in a relevant art.

Features described with respect to certain example embodiments can be combined and sub-combined in and/or with various other example embodiments. Also, different aspects and/or elements of example embodiments, as disclosed herein, can be combined and sub-combined in a similar manner as well. Further, some example embodiments, whether individually and/or collectively, can be components of a larger system, wherein other procedures can take precedence over and/or otherwise modify their application. Additionally, a number of steps can be required before, after, and/or concurrently with example embodiments, as disclosed herein. Note that any and/or all methods and/or processes, at least as disclosed herein, can be at least partially performed via at least one entity in any manner.

Various terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc. can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Various terminology used herein is for describing particular example embodiments and is not intended to comprise necessarily limiting of this disclosure. As used herein, various singular forms “a,” “an” and “the” are intended to include various plural forms as well, unless a context clearly indicates otherwise. Various terms “comprises,” “includes” and/or “comprising,” “including” when used in this specification, specify a presence of stated features, integers, steps, operations, elements, and/or components, but do not preclude the presence and/or addition of one or more other features, integers, steps, operations, elements, components, and/or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

As used herein, the term “stitching” includes any stitching pattern that can connect two or more layers, without being limited thereto. Any such stitching pattern may be seamless, but is not limited thereto.

As used herein, the term “knitting” includes any knitting pattern that can connect two or more layers, without being limited thereto. Any such knitting pattern may be seamless, but is not limited thereto.

As used herein, the term “free cut” means a portion of a garment that represents a border of the garment, without elastic and without stitching along the border. In certain embodiments, “free cut” includes a bonding or taping along the border.

Example embodiments of this disclosure are described herein with reference to illustrations of idealized embodiments (and intermediate structures) of this disclosure. As such, variations from various illustrated shapes as a result, for example, of manufacturing techniques and/or tolerances, are to comprise expected. Thus, various example embodiments of this disclosure should not be construed as necessarily limited to various particular shapes of regions illustrated herein, but are to include deviations in shapes that result, for example, from manufacturing.

Any and/or all elements, as disclosed herein, can be and/or include, whether partially and/or fully, a solid, including a metal, a mineral, an amorphous material, a ceramic, a glass ceramic, an organic solid, such as wood and/or a polymer, such as rubber, a composite material, a semiconductor, a nanomaterial, a biomaterial and/or any combinations thereof. Any and/or all elements, as disclosed herein, can be and/or include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as for tint, color, hue, saturation, tone, shade, transparency, translucency, opaqueness, luminescence, reflection, phosphorescence, anti-reflection and/or holography, a photo-sensitive coating, an electronic and/or thermal property coating, such as for passivity, insulation, resistance or conduction, a magnetic coating, a water-resistant and/or waterproof coating, a scent coating and/or any combinations thereof. Any and/or all elements, as disclosed herein, can be rigid, flexible, and/or any other combinations thereof. Any and/or all elements, as disclosed herein, can be identical and/or different from each other in material, shape, size, color and/or any measurable dimension, such as length, width, height, depth, area, orientation, perimeter, volume, breadth, density, temperature, resistance, and so forth.

Unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized and/or overly formal sense unless expressly so defined herein.

Furthermore, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” and/or “substantially” refers to a +/−10% variation from a nominal value/term. Such variation is always included in any given value/term provided herein, whether or not such variation is specifically referred thereto.

If any disclosures are incorporated herein by reference and such disclosures conflict in part and/or in whole with this disclosure, then to an extent of a conflict, if any, and/or a broader disclosure, and/or broader definition of terms, this disclosure controls. If such disclosures conflict in part and/or in whole with one another, then to an extent of a conflict, if any, a later-dated disclosure controls.

According to an exemplary embodiment of this disclosure, a device comprises: a garment comprising a body, two leg openings, and a crotch area on an inner side of the garment, wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: a garment comprising a body, two leg openings, a material surrounding each of the leg openings and having a length not extending past a knee of a wearer, and a crotch area on an inner side of the garment, wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: a garment comprising a body, two leg openings, a crotch area on an inner side of the garment, and a material surrounding each of the two leg openings and having a length extended past a knee of a wearer, wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: an outer garment surrounding an inner garment, wherein the outer garment extends beyond the inner garment, wherein the inner garment comprises a body, two leg openings, a crotch area on an inner side of the garment, an wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

According to another exemplary embodiment of this disclosure, a device comprises: a garment, wherein the garment comprises a front portion, wherein the front portion comprises a first front portion substantially covering a chest of a wearer, a second front portion substantially covering a torso of a wearer between the chest and a waistline of the wearer, and a third front portion substantially covering the torso of the wearer below the waistline; a rear portion; an inner front elastic portion connecting the first front portion and the second front portion at an inner side of the garment; an inner rear elastic portion across a back of the wearer to connect a first edge and a second edge of the first portion; two leg openings, and optionally, a pair of straps connecting the front portion and the rear portion; wherein the third portion comprises a crotch area, and wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers, wherein at least one of the first to fourth layers comprises a moisture-wicking material or a waterproof material, and wherein each of the first to fourth layers is, independently, a single layer or a plurality of layers.

The device according to any one of the prior exemplary embodiments, wherein the second layer extends between the first layer and the third layer, and the third layer extends between the second layer and the fourth layer.

The device according to any one of the prior exemplary embodiments, wherein a material of each of the first to fourth layers is same or different, and is independently selected from fabric, natural yarn, synthetic yarn, cotton, silk, polyester, spandex, rubber, plastic, metal, merino wool, nylon, polypropylene, rayon, linen, spandex, bamboo, Gore-Tex, X-static, tencel or combinations thereof. The thickness of each of the first to fourth layers may be from about 0.5 mm to about 10 mm, preferably from about 0.6 mm to 9 mm, from about 0.7 mm to about 8 mm, from about 0.8 mm to about 7 mm, from about 0.9 mm to about 6 mm, from about 1 mm to about 5 mm, from about 1.5 mm to about 4.5 mm, from about 2 mm to about 4 mm, from about 3 mm to about 3.5 mm, and more preferably, about 1 mm, about 1.25 mm, about 1.5 mm, about 1.75 mm, about 2.0 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, and any other suitable thickness within these ranges.

The device according to any one of the prior exemplary embodiments, further comprising one or more additional layers between the first and fourth layers.

The device according to any one of the prior exemplary embodiments, wherein two or more of the first to fourth layers are bonded or adhered together via a stitching, a knitting, an adhesive layer or a bonding tape.

The device according to any one of the prior exemplary embodiments, further comprising a fifth layer extending between the fourth layer and the main body, wherein the fifth layer is a single layer or a plurality of layers.

The device according to any one of the prior exemplary embodiments, wherein the second layer and the third layer are separate layers.

The device according to any one of the prior exemplary embodiments, wherein the second layer and the third layer are combined in a single layer.

The device according to any one of the prior exemplary embodiment,, wherein the second layer further comprises a pocket; and wherein the third layer is included inside the pocket.

The device according to any one of the prior exemplary embodiments, wherein the second layer further comprises a pocket; and wherein a plurality of third layers are included inside the pocket.

The device according to any one of the prior exemplary embodiments, wherein the third layer comprises one or more layers.

The device according to any one of the prior exemplary embodiments, wherein a material of each of the one or more layers is same or different, and is independently selected from fabric, natural yarn, synthetic yarn, cotton, silk, polyester, spandex, rubber, plastic, metal, merino wool, nylon, polypropylene, rayon, linen, spandex, bamboo, Gore-Tex, X-static, tencel or combinations thereof. However, material of any one or more of the garment described herein is not limited to comprising the above-described materials, and any suitable material may be incorporated into the garment.

The device according to any one of the prior exemplary embodiments, wherein the moisture-wicking materials is selected from fabric, natural yarn, synthetic yarn, wool, polypropylene, nylon, polyester or combinations thereof. The moisture-wicking material may be any material that allows one-way transport of moisture, water and/or liquids. A layer comprising the moisture-wicking material may be a water/moisture absorbable layer, such as a polymer blend, including but not limited to a polyester/nylon blend (e.g., 80% polyester, 20% nylon). In various exemplary embodiments, the moisture absorbable layer may include a combination of materials such as, but not limited to, 100% polyester, 96% cotton/4% spandex, 80% polyester/20% nylon, Bramante®/cotton blend, cotton flock, natural fibers, wool, and the like. In various exemplary embodiments, the moisture-wicking material may be a combination of materials selected from fabric, natural yarn, synthetic yarn, wool, polypropylene, nylon, polyester or combinations thereof. Various, non-limiting, exemplary embodiments of the moisture-wicking layer are listed in Table 1:

TABLE 1

| Composition | Weight (g) | Thickness (mm) |
|---|---|---|
| 88 Polyester/12 Lyocell | 175 | 120 |
| 45 Polyester/8 Elastane/47 | 305 | 165 |
| Cotton | | |
| 80 Polyester/20 Elastane | 220 | 140 |
| 27 Cotton/73 Polyester | 140 | 160 |
| 68 Supima Cotton/26 | 170 | 165 |
| Polyester/6 Elastane | | |
| 71 Modal/21 Polyester/8 | 160 | 135 |
| Elastane | | |
| 68 Lyocell/ 25 Polyester/7 | 170 | 170 |
| Elastane | | |
| 90 Polyester/10 Elastane | 195 | 125 |
| 96 Micro Modal Air Jet/4 | 160 | 155 |
| Elastane | | |
| 48 Micromodal Compact/41 | 180 | 151 |
| Polyamide/11 Elastane | | |
| 34 Supima Cotton/34 | 205 | 130 |
| Micromodal Air/32 Elastane | | |
| 43 Viscose/39 Micromodal | 200 | 150 |
| Compact/18 Elastane | | |
| 39 Cotton/22 Seacell/34 | 180 | 163 |
| Modal/5 Spandex | | |
| 82.7 Seacell Modal/12.3 | 160 | 164 |
| Polyester/5 Spandex | | |
| 95Ecovero Viscose/5 | 150 | 160 |
| Spandex | | |
| 56 Modal/37 Refibra/7 | 170 | 160 |
| Spandex | | |
| 89 Repreve Recycled | 150 | 147 |
| Polyester/11 Recycled | | |
| Spandex | | |

RANGES OF THICKNESS, WIGHT, MATERIALS (elastine 5-25%; polyester x-y % However, the moisture-wicking material is not limited thereto, and any suitable moisture-wicking material may be incorporated into the garment.

The device according to any one of the prior exemplary embodiments, wherein a shape of the second layer is different from a shape of the third layer.

The device according to any one of the prior exemplary embodiments, wherein a shape of the second layer is the same as a shape of the third layer.

The device according to any one of the prior exemplary embodiments, wherein a size of the third layer is smaller than a size of the second layer such that the second layer extends beyond the third layer.

The device according to any one of the prior exemplary embodiments, wherein each of the first to fourth layers has a first shape, wherein an upper portion and a lower portion having equal dimensions are connected by a middle portion having a narrower width than the upper and lower portions.

The device according to any one of the prior exemplary embodiments, wherein each of the first to fourth layers has a modified shape, wherein the modified shape comprises a first shape portion connected to a second shape portion, wherein the first shape portion comprises an upper portion and a lower portion having equal dimensions connected by a middle portion having a narrower width than the upper and lower portions, and wherein the second shape portion comprises a trapezoidal shape tapering from the first shape portion to a rear edge of each of the first to fourth layers.

However, the shape of any of the layers of the garment described herein is not limited to comprising a specific shape described herein, and other shapes or shape patterns are possible, including but not limited to, triangular, circular, rectangular, square, octagon, pentagon, hexagon, pentagonal or hexagonal star, rhombus, polka dot, and the like.

The device according to any one of the prior exemplary embodiments, wherein each of the first to fourth layers is arranged such that the first shape portion extends towards a front of the garment and the second shape portion extends towards a rear of the garment.

The device according to any one of the prior exemplary embodiments, wherein at least the first layer comprises the moisture-wicking material.

The device according to any one of the prior exemplary embodiments, wherein at least the first layer comprises the waterproof material.

The device according to any one of the prior exemplary embodiments, wherein the stitching, the knitting, the adhesive layer or the bonding tape avoids extending through the material.

The device according to any one of the prior exemplary embodiments, wherein each of the two leg openings is a free cut leg opening.

The device according to any one of the prior exemplary embodiments, wherein an elastic edge surrounds each of the two leg openings.

The device according to any one of the prior exemplary embodiments, wherein an elastic edge surrounds each of the two leg openings, and wherein an edge of each of the two leg openings is folded inwards to enclose the elastic edge.

The device according to any one of the prior exemplary embodiments, wherein an elastic edge portion surrounds a waistband portion of the body.

The device according to any one of the prior exemplary embodiments, wherein the first layer is bonded or adhered to the second layer along outer edges of the first and second layers via a stitching, a knitting, an adhesive layer or a bonding tape.

The device according to any one of the prior exemplary embodiments, wherein the crotch area is bonded to the body via a stitching, a knitting, an adhesive layer or a bonding tape, and wherein the stitching, the knitting, the adhesive layer or the bonding tape extends beyond the crotch area to a waistband edge of the body.

The device according to any one of the prior exemplary embodiments, wherein a grain of the moisture-wicking material is arranged to be longitudinal from a front to a rear portion of the at least one of the first to fourth layers comprising the moisture-wicking material.

The device according to any one of the prior exemplary embodiments, wherein a volume capacity of the one or more layers comprising the moisture-wicking material or waterproof material is in a range of from about 50 ml to about 300 ml, preferably from about 55 ml to about 250 ml, from about 60 ml to about 200 ml, from about 65 ml to about 150 ml, and more preferably, about 70 ml, about 75 ml, about 80 ml, about 85 ml, about 90 ml, about 95 ml, about 100 ml, about 105 ml, about 110 ml, about 115 ml, about 120 ml, about 125 ml, about 130 ml, about 135 ml, about 140 ml, about 145 ml, about 150 ml, about 155 ml, about 160 ml, about 165 ml, about 170 ml, about 175 ml, about 180 ml, about 185 ml, about 190 ml, about 195 ml, about 200 ml, about 205 ml, about 210 ml, about 215 ml, about 220 ml, about 225 ml, about 230 ml, about 235 ml, about 240 ml, about 245 ml, about 250 ml, about 255 ml, about 260 ml, about 265 ml, about 270 ml, about 275 ml, about 280 ml, about 285 ml, about 290 ml, about 295 ml, about 300 ml, and any other suitable volume within these ranges.

The device according to any one of the prior exemplary embodiments, further comprising a material surrounding each of the leg openings and having a length extending past a knee of a wearer.

The device according to any one of the prior exemplary embodiments, further comprising a front waistband pocket located on an inner side of the garment.

The device according to any one of the prior exemplary embodiments, further comprising a temperature pad pocket located on an inner side of the garment.

The device according to any one of the prior exemplary embodiments, further comprising a mesh liner on an inner side of the body, and connected to the body along one edge.

The device according to any one of the prior exemplary embodiments, further comprising a pocket located in an inner side of a waistband portion, wherein the pocket includes a zipper, a self-fastener, an interconnecting fabric connector, a hook and loop fastener, a button fastener, a closure means, or a combination thereof.

The device according to any one of the prior exemplary embodiments, further comprising a front waistband pocket located on an inner side of the garment.

The device according to any one of the prior exemplary embodiments, further comprising a heating pad inside the front waistband pocket.

The device according to any one of the prior exemplary embodiments, further comprising a mesh liner on an inner side of the body, and connected to the body along one edge.

The device according to any one of the prior exemplary embodiments, wherein an outer garment comprises a waist portion, a front first portion, a front second portion, a rear first portion, and a rear second portion, wherein the front first and second portions have a trim along edges thereof.

According to an exemplary embodiment of this disclosure, a method comprises: causing a garment to comprise a body, two leg openings, and a crotch area on an inner side of the garment, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, causing at least one of the first to fourth layers to comprise a moisture-wicking material or a waterproof material, and causing each of the first to fourth layers to be, independently, a single layer or a plurality of layers.

According to another exemplary embodiment of this disclosure, a method comprises: causing a garment to comprise a body, two leg openings, a material surrounding each of the leg openings and having a length not extending past a knee of a wearer, and a crotch area on an inner side of the garment, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, and causing at least one of the first to fourth layers comprises a moisture-wicking material or a waterproof material, and wherein each of the first to fourth layers is, independently, a single layer or a plurality of layers.

According to another exemplary embodiment of this disclosure, a method comprises: causing a garment to comprise a body, two leg openings, a crotch area on an inner side of the garment, and a material surrounding each of the two leg openings and having a length extended past a knee of a wearer, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, causing at least one of the first to fourth layers to comprise a moisture-wicking material or a waterproof material, and causing each of the first to fourth layers to be, independently, a single layer or a plurality of layers.

According to another exemplary embodiment of this disclosure, a method comprises: causing an outer garment to surround an inner garment, wherein the outer garment extends beyond the inner garment, causing the inner garment to comprise a body, two leg openings, a crotch area on an inner side of the garment, causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers, causing at least one of the first to fourth layers to comprise a moisture-wicking material or a waterproof material, and causing each of the first to fourth layers to be, independently, a single layer or a plurality of layers.

According to another exemplary embodiment of this disclosure, a method comprises: causing a garment to comprise a front portion, wherein the front portion comprises a first front portion substantially covering a chest of a wearer, a second front portion substantially covering a torso of a wearer between the chest and a waistline of the wearer, and a third front portion substantially covering the torso of the wearer below the waistline; causing a garment to comprise a rear portion; causing a garment to comprise an inner front elastic portion connecting the first front portion and the second front portion at an inner side of the garment; causing a garment to comprise an inner rear elastic portion across a back of the wearer to connect a first edge and a second edge of the first portion; causing a garment to comprise two leg openings; causing a garment to comprise, optionally, a pair of straps connecting the front portion and the rear portion; causing the third portion to comprise a crotch area; causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers; causing at least one of the first to fourth layers to comprise a moisture-wicking material or a waterproof material; and causing each of the first to fourth layers to be, independently, a single layer or a plurality of layers.

In some embodiments, various functions or acts can take place at a given location and/or in connection with the operation of one or more apparatuses or systems. In some embodiments, a portion of a given function or act can be performed at a first device or location, and a remainder of the function or act can be performed at one or more additional devices or locations.

The corresponding structures, materials, acts, and equivalents of all means or step plus function elements in the claims below are intended to include any structure, material, or act for performing the function in combination with other claimed elements as specifically claimed. The embodiments were chosen and described in order to concisely explain the principles of the disclosure and the practical application, and to enable others of ordinary skill in the art to understand the disclosure for various embodiments with various modifications as are suited to the particular use contemplated.

Various exemplary embodiments of this application are illustrated in the accompanying drawings.

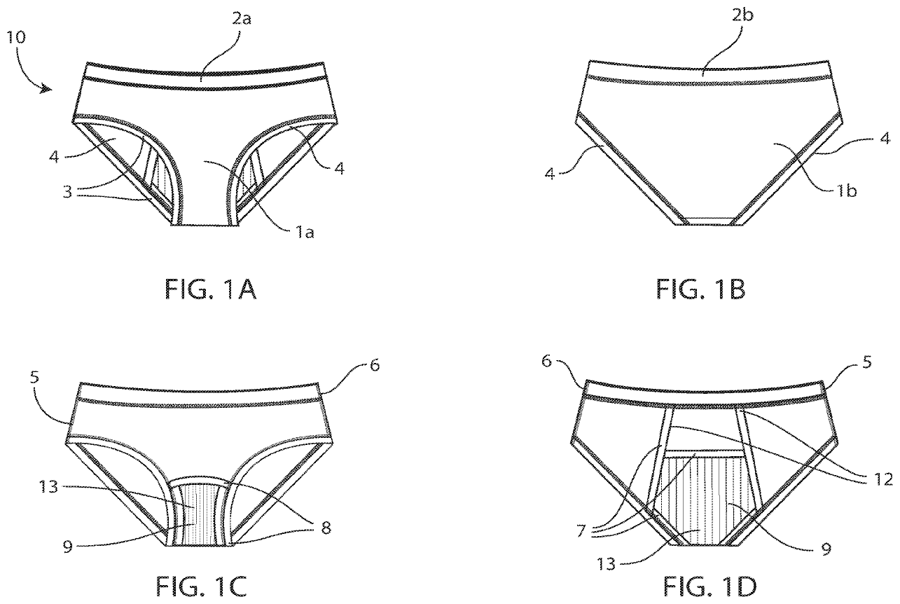

FIGS. 1A to 1D show various perspective views of a garment according to a first exemplary embodiment. FIG. 1A is a front outer view and FIG. 1B is a rear outer view of a garment according to a first exemplary embodiment. As shown in FIGS. 1A and 1B, a garment 10 has a front portion 1a and a rear portion 1b, including an elastic waistband top 2a (front) and 2b (rear) at a waist level of a wearer and two leg openings 4. Each of the two leg openings may be encircled by an elastic layer 3, wherein an elastic layer is sandwiched between fabric layers, wherein the fabric may be folded over the elastic layer. In other embodiments, each of the two leg openings may be a free cut leg opening. FIG. 1C is a front inner view and FIG. 1D is a rear inner view of a garment according to the first exemplary embodiment. As shown in FIGS. 1C and 1D, the garment 10 includes side seams 5 (front view left side and rear view right side) and 6 (front view, right side and rear view, left side) along an inner length of the garment. The sides seams extend through the waistband elastic to create a clean finish when viewed from an outer side.

As shown in FIG. 1C, the gusset 13 is coupled to the front of garment 10 via bonding strips 8. As shown in FIG. 1D, the gusset 13 is coupled to the rear of garment 10 via bonding strips 7, which includes a portion 12 that extends beyond the edge of the gusset 13. In certain embodiments, the gusset 13 is coupled to the garment via stitching. This coupling may be at the front, at the rear, or both at the front and the rear. In certain embodiments, the stitching may extend beyond the edge of the gusset 13. The gusset 13 includes grain lines 9 that are directed longitudinally from the front to the rear of the garment 10. In alternative embodiments the grain lines are positioned in other arrangements, such as horizontally, radially or in any shape or pattern. In other embodiments, there are no grain lines.

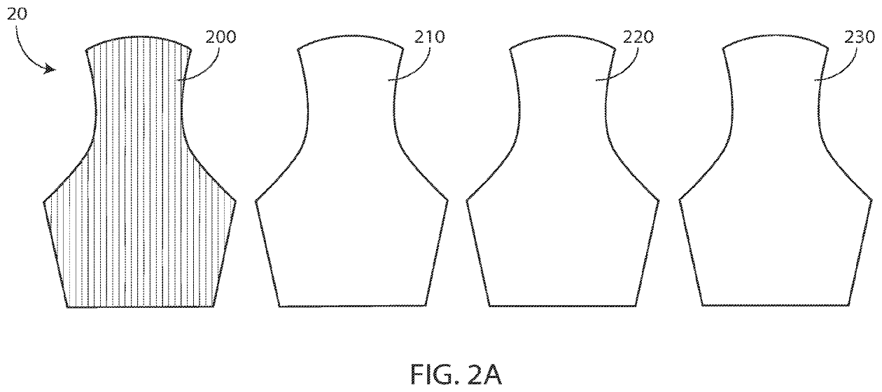

FIGS. 2A and 2B illustrate arrangements of various layers of a gusset 20 according to exemplary embodiments. As illustrated in FIGS. 2A and 2B, a gusset 20 may include a first layer 200, a second layer 210, a third layer 220, a fourth layer 230, and optionally, a fifth layer 240. The gusset 20 may further comprise additional layers in addition to the first to fifth layers, arranged in any combination extending between the first and fifth layer, such as between the first and the second layers, between the first and the third layers, between the first and the fourth layers, between the first and the fifth layers, between the second and the third layers, between the second and the fourth layers, between the second and the fifth layers, between the third and the fourth layers, between the fourth and the fifth layers, and any combination therebetween. The gusset may also include additional layers arranged outside the first to the fifth layers.

In an exemplary embodiment, the first layer 200 may be an innermost layer and the fourth layer 230 may be an outermost layer. In another exemplary embodiment, the first layer may be an innermost layer and the fifth layer 240 may be an outermost layer. In another exemplary embodiment, the first layer 200 may include a moisture-wicking material or a partially waterproof material. In another exemplary embodiment, the fourth layer 230 and/or the fifth layer 240 may include a waterproof material. In another exemplary embodiment, any of the first to fifth layers may be, independently, a single layer or a plurality of layers. In another exemplary embodiment, two or more of the first to fourth layers are bonded or adhered together via a stitching, a knitting, an adhesive layer or a bonding tape.

In another exemplary embodiment, the second layer 210 and the third layer 220 are combined in a single layer, as illustrated in FIG. 2B.

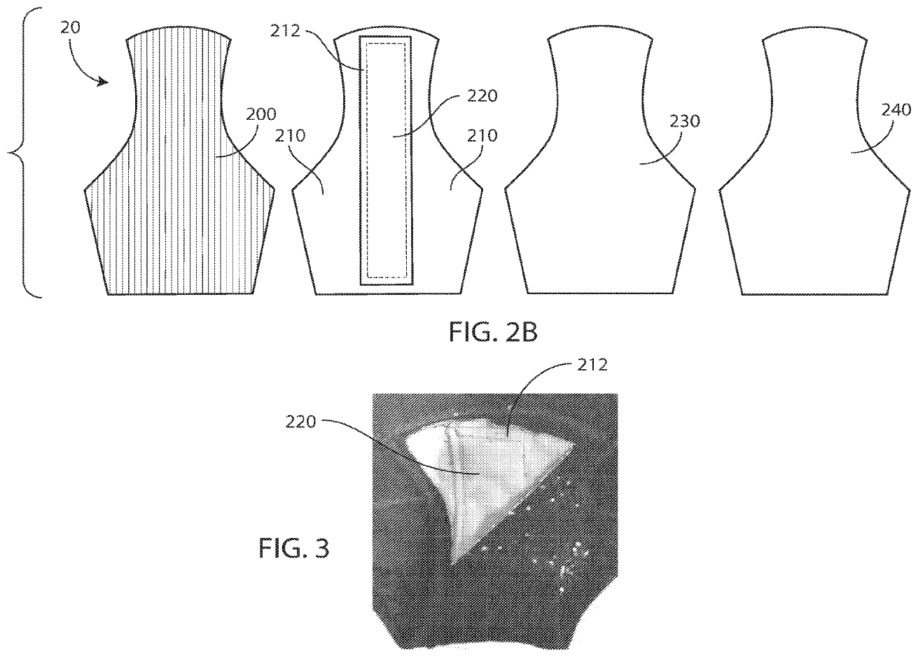

In another exemplary embodiment, as shown in FIG. 2B, the second layer 210 may further comprises a pocket 212, and the third layer 220 may be included inside the pocket 212, as also shown in FIG. 3. In another exemplary embodiment, a plurality of third layers 220 may be included inside the pocket 212. In another exemplary embodiment, the third layer 220 may comprise a single layer or a plurality of layers. In alternative embodiments, the third layer 220 is coupled directly to the second layer 210 or the third layer 220 is positioned adjacent to the second layer 210 without the need for a pocket 212.

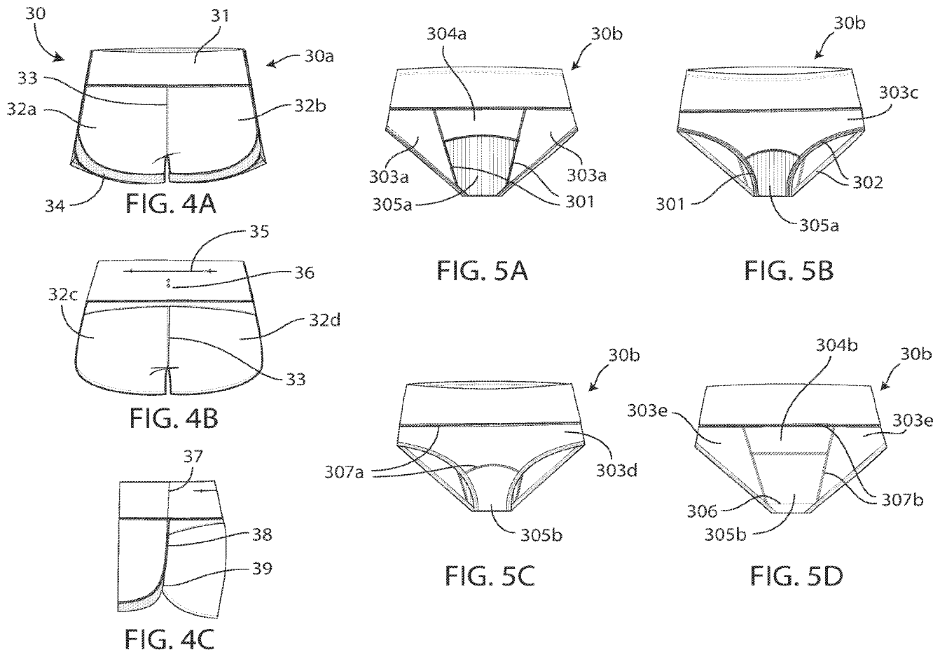

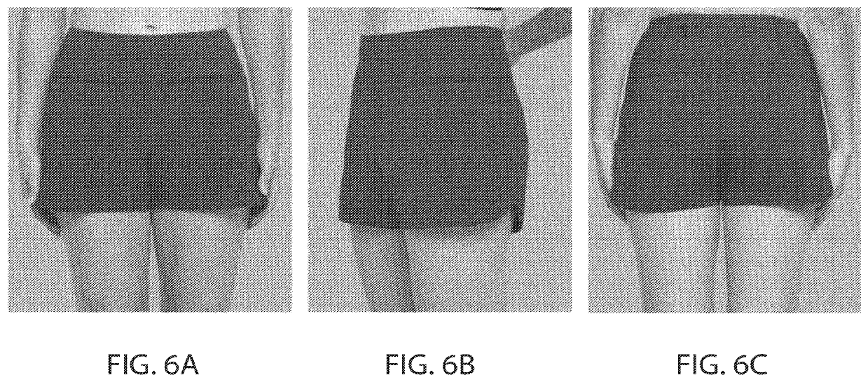

FIGS. 4A to 4C show various perspective (outer) views of a garment according to a second exemplary embodiment, and FIGS. 5A to 5D show various perspective (inner) views of a garment 30 according to a second exemplary embodiment. As shown in FIGS. 4A to 4C and 5A to 5D, a garment according to a second exemplary embodiment includes an outer first garment 30a (FIGS. 4A to 4C) and an inner second garment 30b (FIGS. 5A to 5D). The outer first garment 30a includes a waist band portion 31. It also includes a first outer front portion 32a, and a second outer front portion 32b, which are connected via a stitching 33, and a third outer rear portion 32c, and a fourth outer rear portion 32d, which are connected via stitching 33. A front portion and a rear portion of the garment 30 may be connected via a seam stitching 38, such that the front and rear portions do not overlap. The seam stitching 38 may extend as stitching 37 into the waist band portion 31. In an exemplary embodiment, the seam stitching may create a clean finish when viewed from an outer side. The outer portions 32a and 32b may also include a double layer portion 34 along an outer bottom circumference. The double layer portion 34 may comprise a mesh material or a mesh liner. The double layer portion may finish at a bottom 39 of a side seam.

A garment according to an exemplary embodiment may also comprise an invisible connecting portion 35. In an exemplary embodiment, the invisible connecting portion may be any fastener, including but not limited to a zipper, a self-fastener, an interconnecting fabric connector, a hook and loop fastener, a button fastener, a closure means, and the like. As used herein, the term “invisible” describes any feature that is not visible on an outer-side of a garment, but may be visible on an inner side of a garment. As used herein, the term “invisible” also describes any feature that is not visible on both an outer side of a garment and an inner-side of a garment.

A garment according to an exemplary embodiment may also comprise a logo 36. The logo 36 may be any type of logo known in the art, including but not limited to, an appliqué, a patch, a stitched pattern, a painted pattern, a heat-seal logo, and the like.

As further shown in FIGS. 5A to 5D, the garment according to the second exemplary embodiment also includes an inner portion, which may include a front inner portion and a back inner portion. As shown in FIG. 5A, an inner view of the rear inner portion 30b comprises the waistband portion 31, an inner middle (rear) portion 304a, at least two side portions 303a and 303a, and an inner crotch portion 305a comprising a gusset. The inner crotch portion 305a is surrounded by stitching 301. As shown in FIG. 5B, the two leg openings are surrounded by a stitching 302, which, optionally, may have a fabric portion enclosing the same. Also shown is an inner (front) portion 303c and an inner view of the inner crotch portion 305a, comprising the gusset. As illustrated in FIGS. 5C and 5D, an outer view of the inner portion of the garment 30b may also include an outer middle (rear) portion 304b and an outer crotch portion 305b, outer (front) portion 303d, at least two side portions 303e, seam 306 connecting the front and back portions, and stitching patterns 307a and 307b connecting the various portions of the inner portion of the garment 30.

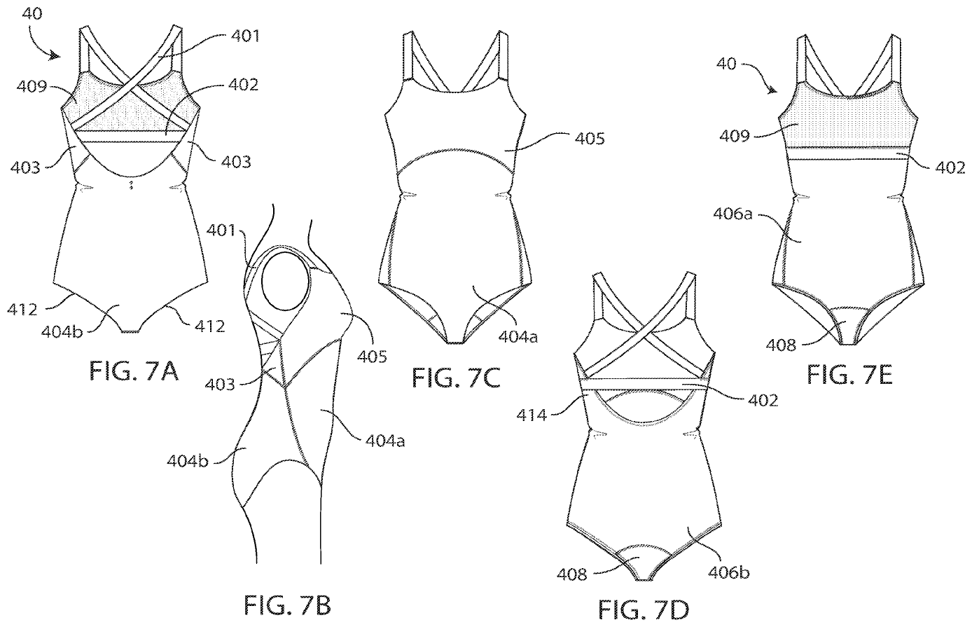

FIGS. 6A and 6B are images of a garment according to the second exemplary embodiment.

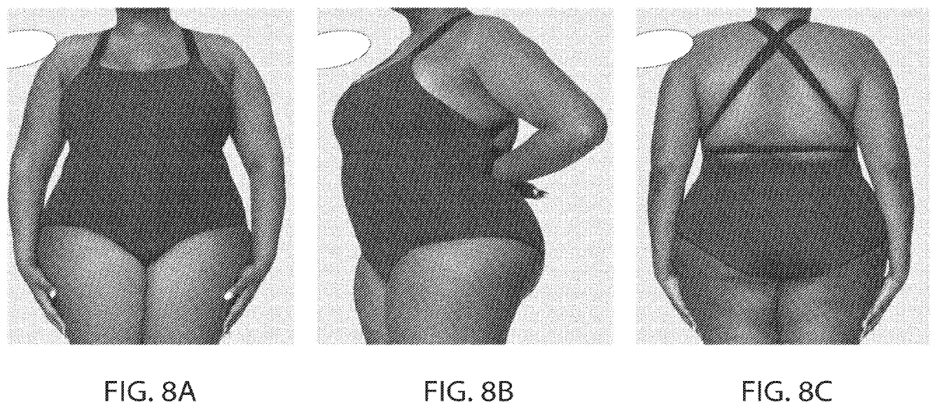

FIGS. 7A to 7E show various perspective views of a garment 40 according to a third exemplary embodiment. As illustrated in FIG. 7C, the garment 40 includes: a first front portion 405 substantially covering a chest of a wearer, and a second outer front portion 404a substantially covering a torso of a wearer between the chest and a waistline of the wearer. FIG. 7A shows a rear portion 404b; an inner elastic portion 402 connected to a bottom of a mesh bra 409 (shown in FIG. 7E), and sewn to an inner back of the garment past upper back outer side panel 403; two leg openings 412; optionally, a pair of straps 401 connecting the top of the front portion 405 and the tops of the rear portion 403. As shown in FIG. 7E, inner front liner portion 406a extends along the entire inner front of the garment 40. Inner elastic portion 402 is connected to the mesh bra 409 that is separate from and overlaps the inner front portion 406a. Also shown is a crotch area 408. The crotch area 408 may comprise a gusset as previously described. FIG. 7D shows the inner back liner portion 406b, and the inner elastic portion 402 which is stitched to the back lining 414 at the top of the inner back portion 406b.

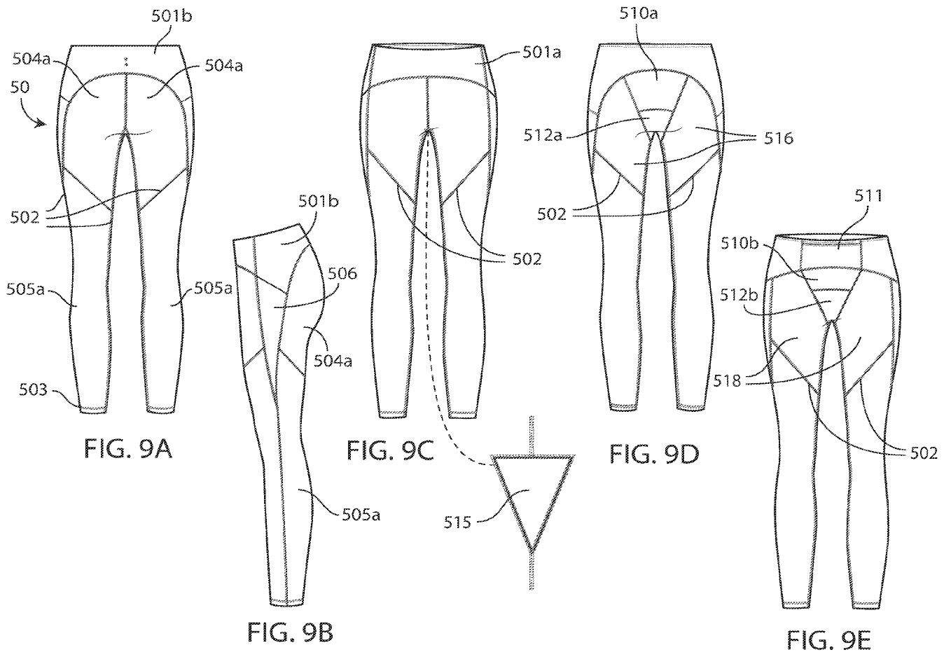

FIGS. 8A to 8C are images of a garment according to the third exemplary embodiment.

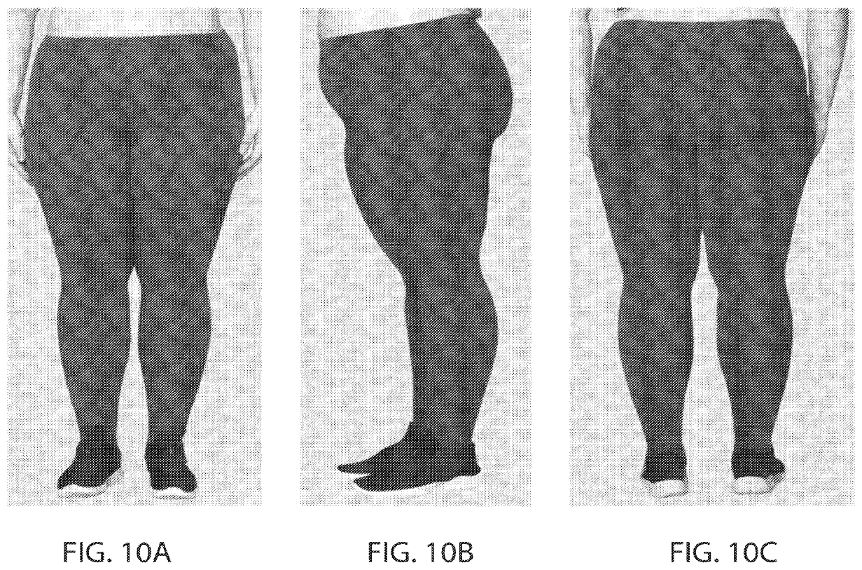

FIGS. 9A to 9E show various perspective views of a garment 50 according to the fourth exemplary embodiment. As shown in FIGS. 9A, 9B and 9C, the garment 50 includes a body, where the body includes front waist band portion 501a, two outer back upper leg panel portions 504a, two outer rear lower leg panel portions 505a, an outer rear waist band portion 501b, stitching 502, which couples an inner liner portion to the thigh portions and the gusset, and a side pocket 506, and a hem portion 503. As shown in FIGS. 9D and 9E, the garment 50 further comprises a rear gusset 512a and a front gusset 512b, which together form a gusset as discussed previously. An inner portion of the garment 50 includes a front inner pocket 511, a front inner crotch liner portion 510b and a rear inner crotch liner portion 510a. As shown in FIG. 9C, The garment also includes a bottom center outer gusset 515. As shown in FIGS. 9D and 9E, an inner portion of the garment includes upper rear liner portions 516 and upper front liner portions 518.

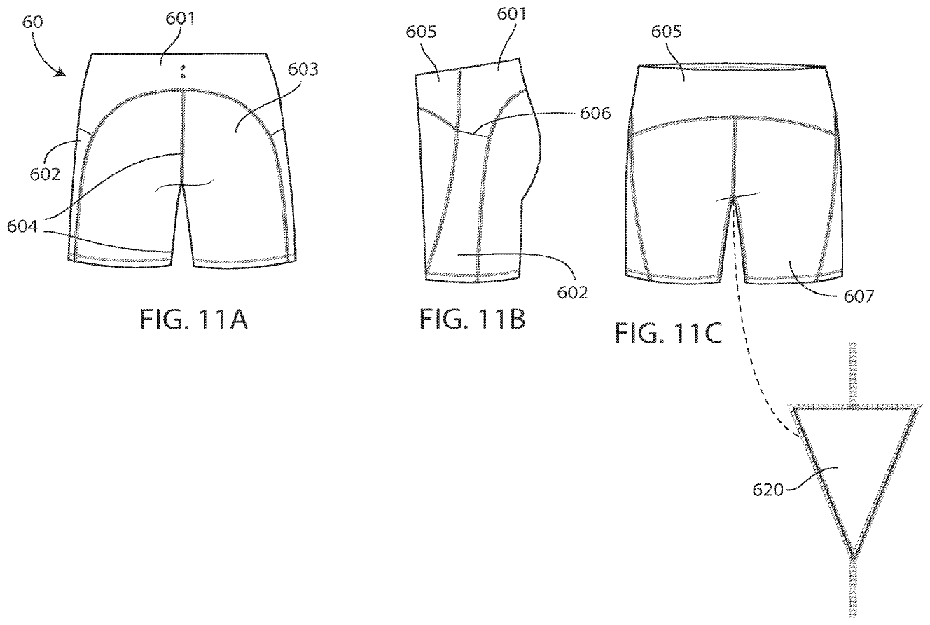

FIGS. 10A to 10C are images of a garment according to a fourth exemplary embodiment.

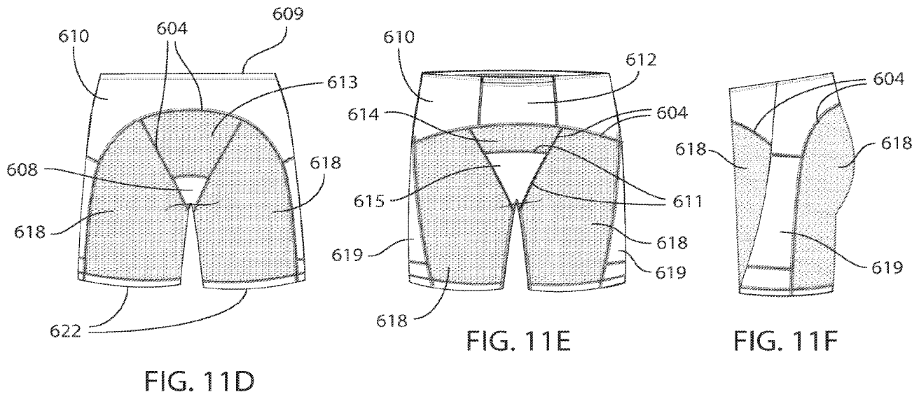

FIGS. 11A to 11F show various perspective views of a garment 60 according to a fifth exemplary embodiment. As shown in FIGS. 11A to 11C, the outside of the garment 60 includes a waist band portion 601, outer side pocket 602, a back outer leg portion 603, and a front outer leg portion 607. The garment 60 also includes an outer gusset 620. The side portion may further include a pocket opening 606, the waist band portion 601, and an upper front panel 605. Each of these portions may be connected via stitching 604. As shown in FIGS. 11D to 11F, the inner portion of garment 60 includes an inner side portion 619, and may further include a mesh layer or mesh liner 618 positioned along the inside of the garment 60 and coupled to the waistband or below the waistband, at an inside of the garment 60. The mesh layer or mesh liner may also be coupled to the garment 60 via the stitching 604, including at or near the leg openings 622. The garment 60 may further, optionally, include a front pocket 612 on an inner side of the waist band portion. The front pocket 612 may optionally include a temperature pad (not shown) to regulate heating/cooling. The inner portion of the garment 60 may also include a rear inner middle portion 613 connecting the waist band portion 610 to the rear crotch (or gusset) portion 608, and a front inner middle portion 614 connecting the waist band portion 610 to the front crotch (or gusset) portion 615, and may further include a stitching pattern 611.

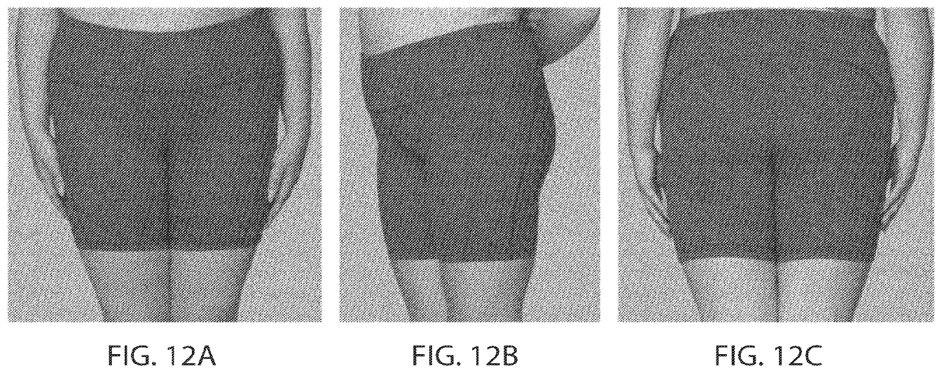

FIGS. 12A to 12C are images of a garment according to a fifth exemplary embodiment.

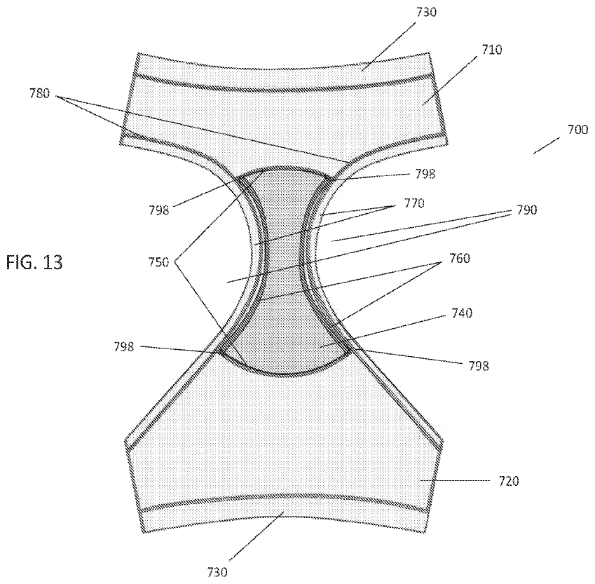

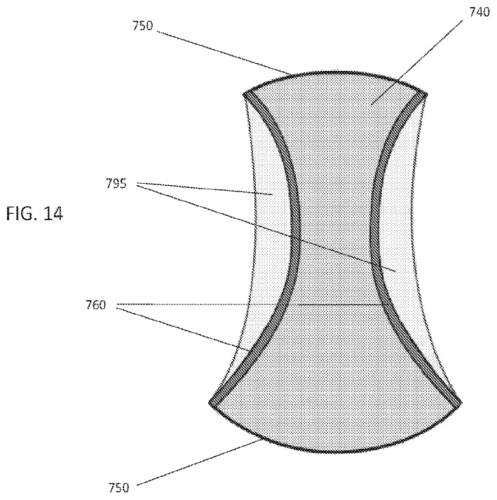

FIG. 13 shows an inside view of a garment, whose side seams have not be sewn, in a laid flat position, according to an exemplary embodiment. FIG. 14 shows an expanded view of the gusset from FIG. 13. As shown in FIG. 13, a garment 700 has a front portion 710 and a rear portion 720, including a front and rear elastic waistband 730 and two leg openings 790. Each of the two leg openings 790 may be encircled by an elastic layer 770, wherein the elastic layer sandwiches the garment fabric 795 (shown in FIG. 14) and includes stitching 780 or some other bonding element, such as, for example, a bonding strip, glue, knitting, welding, or some other bonding agent. In other embodiments, each of the two leg openings may be a free cut leg opening.

As shown in FIGS. 13 and 14, the gusset 740 includes side seams 760 along both sides of the gusset. In certain embodiments, the sides seams are formed from the outermost layer extending past the side edges of the gusset and over the innermost layer. In certain embodiments, the side edges of the fourth layer 230 (as shown in FIG. 2A), which may be an outermost layer, extend over the side edges of the first layer 200. In another exemplary embodiment, the side edges of the fifth layer 240, the outermost layer, extend over the side edges of the first layer 200. The side seams 760 of the gusset, after the outermost layer is extender over the edges of the innermost layer are coupled, using a bonding element, which can be, for example, stitching, a bonding strip, glue, knitting, welding, or some other bonding agent.

As shown in FIGS. 13 and 14, the gusset 740 is coupled to the garment at an edge of the front portion 710 and at an edge of the rear portion 720, via bonding elements 750, which can be, for example, stitching, a bonding strip, glue, knitting, welding, or some other bonding agent. The gusset may also include bartacks 798 at the corners of the front and rear edges of the gusset to provide extra support (as shown in FIG. 13). The gusset 740 is not coupled to the garment at the side edges of the gusset 740 to allow the gusset to float. This allows for space between the gusset 740 and the garment crotch area 795 for insertion of a removable or replaceable absorbable pad or absorption structure for extra absorption.

Any diagrams, images and drawings depicted herein are illustrative. There can be many variations to the diagram or the steps (or operations) described therein without departing from the spirit of the disclosure. For instance, the steps can be performed in a differing order or steps can be added, deleted or modified. All of these variations are considered a part of the disclosure. It will be understood that those skilled in the art, both now and in the future, can make various improvements and enhancements which fall within the scope of the claims which follow.

The description of this disclosure has been presented for purposes of illustration and description, but is not intended to comprise fully exhaustive and/or limited to the disclosure in the form disclosed. Many modifications and variations in techniques and structures will be apparent to those of ordinary skill in an art without departing from a scope and spirit of this disclosure as set forth in the claims that follow. Accordingly, such modifications and variations are contemplated as being a part of this disclosure. A scope of this disclosure is defined by various claims, which include known equivalents and unforeseeable equivalents at a time of filing of this disclosure.

1.-66. (canceled)

67. A device comprising:
a garment comprising a body, two leg openings, a crotch area on an inner side of the garment, and a material surrounding each of the two leg openings and having a length extended past a knee of a wearer,
wherein the crotch area comprises: a first layer, a second layer, a third layer and a fourth layer, wherein the first layer is an innermost layer, wherein the fourth layer is an outermost layer, and wherein the second and third layers are intermediate layers extending between the first and fourth layers,
wherein at least one of the first to fourth layers comprises a moisture-wicking material or a waterproof material, and
wherein each of the first to fourth layers is, independently, a single layer or a plurality of layers.

68. The device of claim 67, wherein the second layer extends between the first layer and the third layer, and the third layer extends between the second layer and the fourth layer.

69. The device of claim 67, wherein a material of each of the first to fourth layers is same or different, and is independently selected from fabric, natural yarn, synthetic yam, cotton, silk, polyester, spandex, rubber, plastic, metal, merino wool, nylon, polypropylene, rayon, linen, spandex, bamboo, Gore-Tex, X-static, tencel, or combinations thereof.

70. The device of claim 67, further comprising one or more additional layers between the first and fourth layers.

71. The device of claim 67, wherein two or more of the first to fourth layers are bonded or adhered together via a stitching, a knitting, an adhesive layer or a bonding tape.

72. The device of claim 67, further comprising a fifth layer extending between the fourth layer and the main body, wherein the fifth layer is a single layer or a plurality of layers.

73. The device of claim 67, wherein the second layer and the third layer are separate layers.

74. The device of claim 73, wherein the second layer and third layer are bonded or adhered together via a stitching, a knitting, an adhesive layer or a bonding tape.

75. The device of claim 67, wherein the second layer and the third layer are combined in a single layer.

76. The device of claim 73, wherein the second layer further comprises a pocket; and wherein the third layer is included inside the pocket.

77. The device of claim 73, wherein the second layer further comprises a pocket; and wherein a plurality of third layers are included inside the pocket.

78. The device of claim 73, wherein the third layer comprises one or more layers.

79. The device of claim 78, wherein a material of each of the one or more layers is same or different, and is independently selected from fabric, natural yarn, synthetic yam, cotton, silk, polyester, spandex, rubber, plastic, metal, merino wool, nylon, polypropylene, rayon, linen, spandex, bamboo, Gore-Tex, X-static, tencel, or combinations thereof.

80. The device of claim 67, wherein the moisture-wicking materials is selected from fabric, natural yarn, synthetic yam, wool, polypropylene, nylon, polyester, or combinations thereof.

81. The device of claim 67, wherein a shape of the second layer is different from a shape of the third layer.

82. The device of claim 67, wherein a shape of the second layer is the same as a shape of the third layer.

83. The device of claim 67, wherein a size of the third layer is smaller than a size of the second layer such that the second layer extends beyond the third layer.

84. The device of claim 67, wherein each of the first to fourth layers has a first shape, wherein an upper portion and a lower portion having equal dimensions are connected by a middle portion having a narrower width than the upper and lower portions.

85. The device of claim 67, wherein each of the first to fourth layers has a modified shape, wherein a first shape portion is connected to a second shape portion, wherein the first shape portion comprises an upper portion and a lower portion having equal dimensions connected by a middle portion having a narrower width than the upper and lower portions, and wherein the second shape portion comprises a trapezoidal shape tapering from the first shape portion to a rear edge of each of the first to fourth layers.

86. The device of claim 67, wherein each of the first to fourth layers is arranged such that the first shape portion extends towards a front of the garment and the second shape portion extends towards a rear of the garment.

87. The device of claim 67, wherein at least the first layer comprises the moisture-wicking material.

88. The device of claim 67, wherein at least the first layer comprises the waterproof material.

89. The device of claim 71, wherein the stitching, the knitting, the adhesive layer or the bonding tape avoids extending through the material.

90. The device of claim 67, wherein each of the two leg openings is a free cut leg opening.

91. The device of claim 67, wherein an elastic edge surrounds each of the two leg openings.

92. The device of claim 67, wherein an elastic edge surrounds each of the two leg openings, and wherein an edge of each of the two leg openings is folded inwards to enclose the elastic edge.

93. The device of claim 67, wherein an elastic edge portion surrounds a waistband portion of the body.

94. The device of claim 67, wherein the first layer is bonded or adhered to the second layer along outer edges of the first and second layers via a stitching, a knitting, an adhesive layer or a bonding tap.

95. The device of claim 67, wherein the crotch area is bonded to the body via a stitching, a knitting, an adhesive layer or a bonding tape, and wherein the stitching, the knitting, the adhesive layer or the bonding tape extends beyond the crotch area to a waistband edge of the body.

96. The device of claim 67, wherein a grain of the moisture-wicking material is arranged to comprise longitudinal from a front to a rear portion of each of the first to fourth layers comprising the moisture-wicking material.

97. The device of claim 67, wherein a volume capacity of the one or more layers comprising the moisture-wicking material or waterproof material is in a range of from about 50 ml to about 300 ml.

98. The device of claim 67, further comprising a front waistband pocket located on an inner side of the garment.

99. The device of claim 98, further comprising a mesh liner on an inner side of the body, and connected to the body along one edge.

100.-164. (canceled)

165. A method comprising:
causing a garment to comprise a body, two leg openings, a crotch area on an inner side of the garment, and a material surrounding each of the two leg openings and having a length extended past a knee of a wearer,
causing the crotch area to comprise: a first layer, a second layer, a third layer and a fourth layer, wherein the first layer is an innermost layer, wherein the fourth layer is an outermost layer, and wherein the second and third layers are intermediate layers extending between the first and fourth layers,
causing at least one of the first to fourth layers to comprise a moisture-wicking material or a waterproof material, and
causing each of the first to fourth layers to comprise, independently, a single layer or a plurality of layers.

166.-172. (canceled)
